(19) United States
(12) Patent Application Publication
(10) Pub. No.: US 2003/0014119 A1
(43) Pub. Date: Jan. 16, 2003

(54) Shoulder prothesis

(76) Inventors: Didier Capon, Sautron (FR); Phlippe Delince, Lasne (BE); Lieven de Wilde, Gand (BE); Pierre Desmoineaux, Le Chesnay (FR); Philippe Dumontier, Yzeure (FR); Denis Katz, Ploemeur (FR); Etienne Lesur, Bayon (FR); Didier Oudet, St Cyr Sur Loire (FR); Anne-Celine Godest, Lyon (FR)

Correspondence Address: YOUNG & THOMPSON, 745 SOUTH 23RD STREET 2ND FLOOR, ARLINGTON, VA, 22202

(21) Appl. No.: 10/175,072

(22) Filed: Jun. 20, 2002

(30) Foreign Application Priority Data
Jun. 21, 2001 (FR) 01 08207

(51) Int. Cl. A61F 2/40

(52) U.S. Cl. 623/19.11

(57) ABSTRACT

The subject of the invention is a shoulder prosthesis (5, 32) comprising a humerus rod (9, 39) intended to be introduced into the medullary cavity of a fractured humerus (2), and a roughly hemispherical head (12) which can be secured to the humerus rod and is designed to become lodged in a glene of the shoulder, characterized in that the humerus head (12) can be fixed to the humerus rod in any radial plane with respect to the axis of the humerus rod (9, 39) after the rod (9, 39) has been fixed into the humerus and the shoulder muscles have been fixed to the head.

The subject of the present invention is a shoulder prosthesis comprising a humerus rod intended to be introduced into the medullary cavity of a fractured humerus, a metaphysal part secured to the rod and a roughly hemispherical head secured to the metaphysal part, designed to become lodged in a glene of the shoulder.

It is known that, following a fracture, the upper end of a humerus may be fractured into several fragments (four or more).

The problems posed by such a multi-fragment fracture were particularly difficult to solve. However, document EP 0 940 126 has described an extremely effective means which allows the tuberosities of the cuff to be fixed on a humerus head of a prosthesis under conditions that allow natural reestablishment of the retroversion of the head and which reestablish normal tension in the various elements of the cuff, while at the same time allowing the arm length to be reestablished.

Document EP 0 940 126 thus describes a shoulder prosthesis in which the humerus head is secured to a shank introduced into a bore in the metaphysal part, inclined with respect to the longitudinal axis thereof. A screw fitted with a stud, screwed axially into the metaphysal part, locks in place the shank secured to the head through the introduction of the stud into a slot in the shank. To fit this prosthesis, the muscles are first of all fixed to the head, then the head is mounted on the rod, which is then immobilized using the screw.

In fact, the surgeon runs into serious difficulties in this process, because he has to place the head on the humerus rod over the field of operation, in which he is unsighted. Furthermore, given the position of the head with respect to the end of the metaphysal part, the immobilizing screw is very difficult to fit. Finally, this arrangement has the disadvantage of concentrating all the load on the screw-locking stud, and this particularly weakens the prosthesis.

At the end of the day, a shoulder prosthesis of the type defined hereinabove needs to meet a certain number of requirements:

it needs to have an epiphysal and metaphysal morphology that allows the anatomical reconstruction of the offset of the posterior overhang and of the retrotorsion of the upper end of the humerus,

it needs to have a morphology that allows the reconstruction and anatomical maintenance of the humerus tuberosities,

it needs to offer the possibility, in respect of all or some of its constituent parts, of refixing the rotators cuff and of maintaining over time the consolidation of the tuberosities.

To allow reconstruction at an appropriate height of the humerus, the rod of the prosthesis has to be correctly positioned heightwise, that is to say that it needs to be placed neither too high up nor too low down.

Documents FR 2 579 454 and 2 699 400 have already proposed shoulder prostheses in which the humerus head can be fixed to the humerus rod in any radial plane with respect to the axis of the humerus rod in order to compensate for the fact that the humerus rods in these documents are fixed in the humerus in some random angular position, but these prostheses are not suited to the problem solved by the invention.

According to the present invention, the prosthesis comprises, on the one hand, a humerus head and, on the other hand, a humerus rod intended to be fixed axially in the humerus, and is characterized in that the humerus head can be fixed on the humerus rod in any radial plane with respect to the axis of the humerus rod, after the humerus rod is fixed in the humerus.

As a preference, the humerus head is secured, for example by being moulded integrally with it, to a metaphysal part which can be assembled with the humerus rod in any angular position.

As a preference, the means of fixing on the humerus rod comprise an assembly of male and female elements, allowing fixing in any relative angular position whatsoever.

Any relative angular position whatsoever is preferably to be understood as meaning an axial assembly, that is to say one along the axis of the humerus rod, with fixing means that allow fixing in any angular position about this axis. However, as a lesser preference, it is possible to envisage for the angular position to be determined by indexing means, provided that the angle between two consecutive indexing means is small.

It is thus possible, once the humerus head has been fixed to the various elements of the rotators cuff, to allow the head to adopt its natural angular position and then, the rod having been fixed into the humerus, to secure them, regardless of the relative angular position of the humerus head and of the rod.

As a particular preference, the humerus head comprises means of fixing the elements of the cuff of the rotators, as defined in European application EP -0 940 126.

These means can be distributed at the periphery of the articular surface of the prosthetic head, for example in the form of holes, allowing shoulder muscle elements to be fixed while the tuberosities or their fragments can be fixed underneath on the head.

Thus, the prosthesis can be made in at least two parts, namely the humerus rod on the one hand, the metaphysal part and the humerus head on the other hand, it being possible for these two parts to be assembled by means of a male-female system, for example of the Morse taper type.

According to one embodiment of the invention, the metaphysal part delimits a female part in the form of a terminal conical socket, while the associated proximal end of the rod consists of a complementary male conical part designed to be introduced into the socket, these two parts being coaxial with the longitudinal axis of the rod.

According to an additional characteristic, the fixing means comprise a screw designed to be screwed into a corresponding tapped hole formed axially in the male conical part, means being provided to allow the screw to be introduced into the said tapped hole.

To fix this prosthesis, the surgeon first of all positions the humerus rod at the appropriate height in the medullary cavity of the humerus then seals it into the humerus. He then fixes the shoulder muscles, particularly the rotators cuff, on the head, using threads. Next, the surgeon positions the fractured bone parts on the head. He then mounts the humerus head on the ready-sealed rod, without permanently fixing it, so as to be able to make retroversion adjustments. Next, he permanently, by impaction, fixes the head on the rod. Finally, he laces the bony fragments on the head and possibly permanently tensions the threads that fix the cuff on the head.

It will be appreciated that it is thus possible, as described in the aforementioned European application, to obtain an ideal position of retroversion of the humerus head and an optimal angular position of the humerus rod in the humerus for the fixing of this rod, which may incidentally have an external surface, particularly towards the metaphysis, which does not exhibit symmetry of revolution, and which can be very well suited to the medullary cavity.

In consequence, it is easy to obtain perfect angular positioning of the humerus head in its anatomical position, taking account of the muscles of the cuff and the stresses exerted on the fractured tuberosities are reduced or eliminated, which fractured tuberosities can knit far more easily, particularly by bone growth, onto the prosthetic head, under the articular surface.

Another subject of the invention is the use of an assembly of a humerus rod and of a humerus head which can be fixed to the rod in any radial plane with respect to the axis of the humerus rod, after the humerus rod has been fixed in the humerus, for the manufacture of a prosthesis equipped with means of fixing bony fragments on the periphery of the head, and intended for the prosthetic treatment of multi-fragment fractures of the upper end of the humerus.

Another subject of the invention is a set of prostheses containing a number of humerus prostheses of different dimensions and/or of humerus head prostheses of different dimensions.

As a preference, use is made of try-in prostheses which have a humerus head and an axial shim and are intended to come into contact with an edge of the humerus fracture and the humerus rod is sealed in the position determined by the shim of the try-in prosthesis.

Another subject of the invention is the try-in prostheses thus defined and a set of these try-in prostheses.

Other particulars and advantages of the invention will become apparent in the course of the description which will follow, given with reference to the appended drawings which illustrate by way of non-limiting examples, several forms of embodiment thereof.

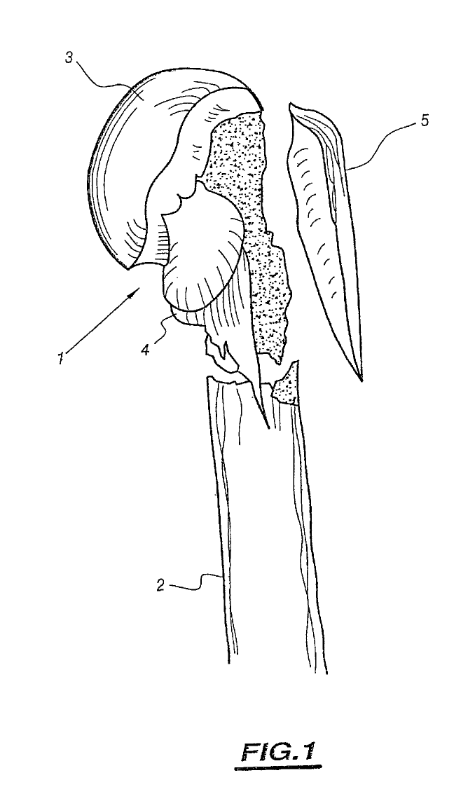

FIG. 1 is a simplified front elevation of the fractured upper end of a humerus.

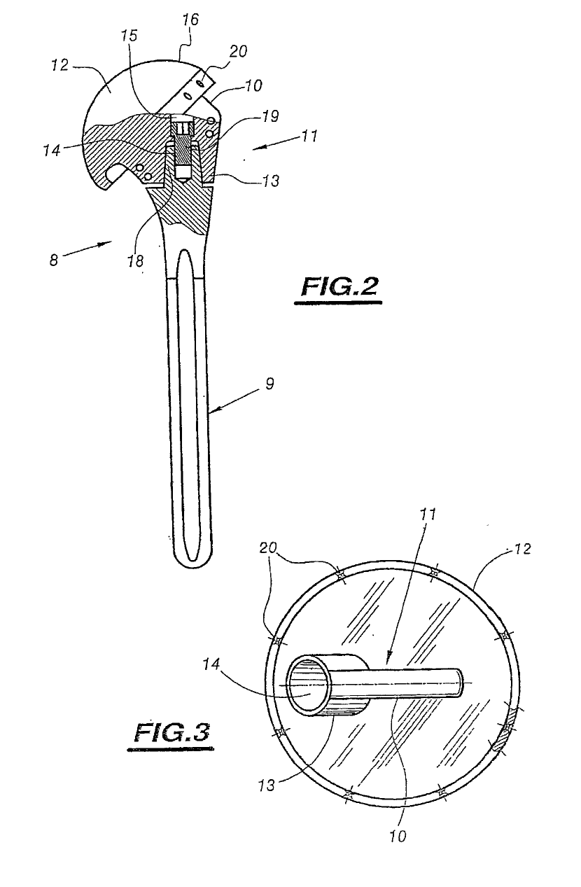

FIG. 2 is a view in partially exploded longitudinal section, of a first form of embodiment of the shoulder prosthesis according to the invention.

FIG. 3 is a plan view on arrow 3 of the metaphysal part and of the head of the prosthesis of FIG. 2.

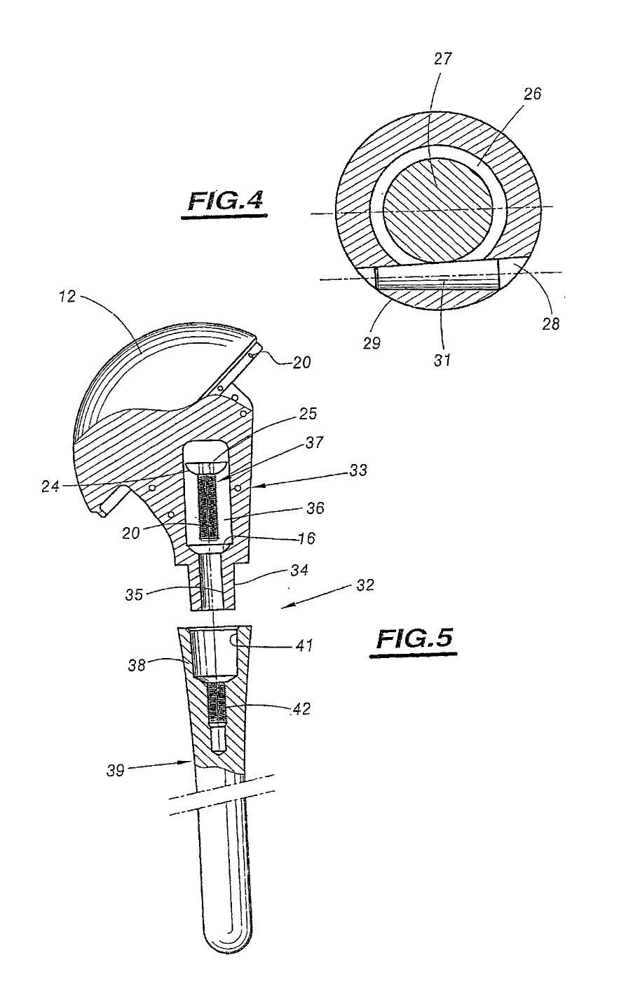

FIG. 4 is a view in cross section on a larger scale than the previous figures, of a second form of embodiment of the prosthesis according to the invention, the section being made at the assembly between the end of the humerus rod and the metaphysal part.

FIG. 5 is a view in longitudinal section of a third form of embodiment of the prosthesis of the invention.

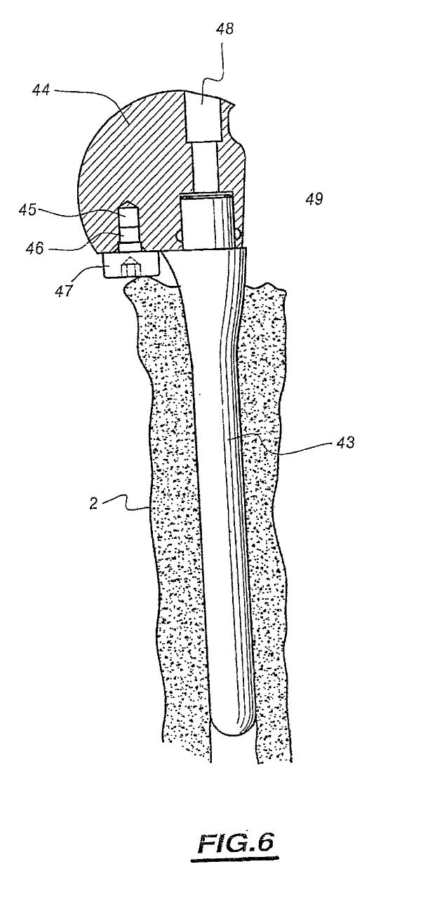

FIG. 6 is a view of a try-in prosthesis of the invention.

FIG. 1 shows the fractured upper end 1 of a humerus 2 with the humerus head 3 detached and shifted with respect to the rest of the humerus, and with respect to the tuberosities 4 and 5, to the glenoid cavity and to the shoulder blade.

The humerus prosthesis at which the invention is aimed has the function of allowing the humerus 2 to be reconstructed heightwise and its end reinserted into the glenoid cavity by capping the head 3 with the various surrounding muscles which have not been depicted.

In its first form of embodiment (FIGS. 2 and 3), the shoulder prosthesis 8 comprises a humerus rod 9 intended to be introduced into the medullary cavity of the fractured humerus 2, a metaphysal part 11 and an epiphysal part 12. The latter consists of a roughly hemispherical head designed to become lodged in the glene. The humerus rod 9 is secured to the metaphysal part 11 by an assembly of male and female elements supplemented by a system for mutually fixing the said elements, the metaphysal part 11 being, for its part, formed of one piece with the head 12.

More specifically, in the embodiment depicted, the metaphysal part 11 comprises a wing 10 running in a diametral plane of the head 12 and a female part 13 in the form of a terminal conical socket formed integrally with the wing 10. The socket 13 internally delimits a female cone 14 the largest diameter of which is at the opposite end to the head 12. This female cone 14 is extended axially by a plain cylindrical hole which itself opens into a hole 15 of larger diameter, so as to form an annular transverse shoulder around the opening into the hole 15. The hole 15 opens freely into the non-articular upper part 16 of the head 12.

The associated proximal end 18 of the rod 9 consists of a male conical part that complements the female conical part 14 and can therefore be introduced into the socket 13.

The means of fixing together the male part 18 and the female part 13 comprise a screw 19 the threaded shank of which is designed to be screwed into a corresponding tapped hole formed axially in the male conical part 18. The screw 19 comprises a head which can be screwed using a tool introduced into the hole 15 to butt up against the shoulder of the hole 15 and clamp the conical bearing surfaces together.

In the second embodiment of the prosthesis, which embodiment is illustrated in FIG. 4, a circular groove 26 is arranged around the conical male part 27, coaxially with respect to it. Furthermore, a conical transverse bore 28 is made in the corresponding female part 29 and opens into the groove 26 roughly tangentially. The bore 28 passes right through the female part 29 and is designed to accommodate a conical pin 31 which can be placed in the position illustrated in FIG. 4, in which it passes through the groove 26. In this position, the pin 31 locks the female 29 and male 27 parts together.

It should however be pointed out that the fitting of a prosthesis according to FIG. 4 may raise difficulties, because the humerus rod 9 is entirely pushed into the fractured humerus 2, which means that the pin 31 would first have to pass through tissue.

In the third form of embodiment illustrated in FIG. 5, the shoulder prosthesis 32 comprises a metaphysal part 33 which is still produced as one piece with the head 12 but which delimits a conical male part 34 in which a plain bore 35 is arranged. The latter opens into an elongate opening 36 formed in the plate which makes up the metaphysal part 33 and having a length that slightly exceeds the length of the locking screw 37.

The proximal end 38 of the humerus rod 39 consists of a female part forming a conical housing 41 designed to accommodate the male part 38. The male 34 and female 38 parts can be fitted together by the screw 37 which is designed to be introduced into the axial bore 35 of the male part 34 and to be screwed into a tapped hole 42 arranged in the proximal end 38 of the rod 39, axially beyond the conical housing 41. The opening 36 in the metaphysal part 33 allows the screw 37 to be introduced into the bore 35 and the hole 42.

In this embodiment, the length needed for the screw 37 exceeds that needed for the screw 19 in the embodiment of FIGS. 2 and 3.

In the three forms of embodiment illustrated in FIGS. 2 to 5, the shoulder prosthesis consists of two parts. As an alternative, it is possible to produce the metaphysal part 11 or 33 and the head 12 in two separate parts which can be assembled by appropriate means, the prosthesis then being made in three parts. Once the humerus rod 9, 39 has been fitted into the fractured humerus, the surgeon fits the metaphysal part on the rod, then fixes the head 12 on the metaphysal part.

It will be understood that, in each form of embodiment, the metaphysal part can be secured to the head with any axial orientation of the head about the longitudinal axis of the metaphysal part or rod.

In order to be able to accommodate the elements of the cuff of the rotator muscles, the spherical part of the head 12 has, at its periphery, means of fixing the cuff, preferably in the form of a number of holes 20 through which threads can be passed to fix the cuff onto the head.

For the various possible modes of embodiment of the prosthesis according to the invention, the surgeon first of all fits the humerus rod, then fixes on the head 12, using threads, the muscles, particularly the rotators cuff. Finally, the surgeon caps the fractured bone parts with the head 12, and fits the metaphysal part onto the proximal end of the rod. If the head 12 and the metaphysal part are separate, the metaphysal part is first of all fitted, followed by the head fixed to the metaphysal part.

For the assembly, reference is now made to FIG. 6 which depicts the two elements of a try-in prosthesis.

The try-in prosthesis comprises a diaphysal try-in rod 43 and a try-in head 44. The try-in head has, on its underside which is perpendicular to the geometric axis of the prosthesis, a tapped hole 45 into which can be screwed a shim 46, the head 47 of which bears against this underside, while remaining outside the try-in prosthesis. The head 47 has a determined thickness. An orifice 48 allows manipulation using an ancillary.

Finally, the head 44 has means 49 allowing it to be mounted on and removed from the rod 43 of the try-in prosthesis and on the rods 9 of the prostheses according to the invention with equal ease.

In order to choose the try-in prosthesis, X-rays of the left arm and right arm are taken, preferably in the pre-operative phase. The two X-rays are then superposed, as precisely as possible. The upper edge of the humerus fracture will then be taken as the point of reference and a shim 46 of a thickness that will allow the upper end of the try-in head to coincide with the upper end of the humerus head of the healthy arm will then be chosen, in advance.

During the operation, after appropriately boring out the diaphysal shaft, the surgeon fits the try-in prosthesis, with the shim 46 determined in that way. He pushes the try-in diaphysal rod 43 into the shaft of the humerus and checks that, when its try-in head is fitted, it lies in the correct position when the shim is pressed against the upper edge of the humerus fracture. If need be, he replaces the shim with another shim of a different thickness.

He thus knows the length that the prosthesis needs to have and the length of the part of the other prosthesis which needs to emerge beyond the humerus fracture.

He then fits the usual plug into the diaphysal shaft. He mounts the head 44 of the try-in prosthesis on the permanent diaphysal part and introduces this into the humerus shaft until once again the abutment piece butts against the upper edge of the line of the fracture of the humerus. The diaphysal part thus finds itself permanently fixed. He then removes the try-in prosthesis. He then assembles the permanent humerus head with the cuff of the rotators using the fixing threads, as described in European Application EP 0 940 126. He then introduces the male axial part of the prosthesis into the corresponding female axial part, for example, the continuation 18 into the orifice 13, gently, so that the head is still able to turn about the diaphysal part. He can then leave the head to be orientated by the cuff of the rotators into the anatomical retroversion position. He checks this position. If it does not suit, he places the suture threads in the holes 20 which are most appropriate.

He then impacts and definitively screws the head onto the diaphysal part in the angular position thus obtained.

He may then complete the intervention by closing up the overlying anatomical planes.

1. Shoulder prosthesis (5, 32), comprising a humerus rod (9, 39) intended to be introduced into the medullary cavity of a fractured humerus (2), and a roughly hemispherical head (12) which can be secured to the humerus rod and is designed to become lodged in a glene of the shoulder, characterized in that the humerus head (12) has means (20) of fixing the shoulder muscles, particularly elements of the cuff, in addition to fixing bony fragments or tuberosities on the head, and in that the head can be fixed to the humerus rod in any radial plane with respect to the axis of the humerus rod (9, 39), once the rod (9, 39) has been fixed into the humerus and the muscles, particularly the rotators cuff, have been fixed to the head.

2. Shoulder prosthesis (5, 32) comprising a humerus rod (9, 39) intended to be introduced into the medullary cavity of a fractured humerus (2), having a shape which does not exhibit symmetry of revolution, at least towards its metaphysal end, so as to be suited to the medullary cavity, and a roughly hemispherical head (12) which can be secured to the humerus rod and is designed to become lodged in a glene of the shoulder, characterized in that the humerus head (12) can be fixed to the humerus rod in any radial plane with respect to the axis of the humerus rod (9, 39) after the rod (9, 39) has been fixed into the humerus and the muscles, particularly the rotators cuff, have been fixed to the head.

3. Prosthesis according to claim 2, characterized in that the humerus head comprises means (20) of fixing the shoulder muscles, particularly elements of the humerus cuff, in addition to fixing bony fragments or tuberosities on the head.

4. Prosthesis according to claim 1, characterized in that these fixing means (20) are distributed on the periphery of the humerus head.

5. Prosthesis according to claim 1, characterized in that these fixing means are peripheral holes (20).

6. Prosthesis according to claim 1, characterized in that the humerus head is secured to a metaphysal part (11, 13) which can be assembled with the humerus rod in any angular position.

7. Prosthesis according to claim 1, characterized in that the humerus head forms a separate part from a metaphysal part capable of being assembled with the humerus head in any angular position.

8. Prosthesis according to claim 1, characterized in that the means of fixing on the humerus head comprise an assembly of male and female elements (13, 18, 34, 38) allowing fixing, after the head has been positioned, in any relative angular position whatsoever.

9. Prosthesis according to claim 1, characterized in that the humerus head and the humerus rod are assembled by means of a male-female system, particularly of the Morse taper type.

10. Prosthesis according to claim 9, characterized in that a metaphysal part (11) secured to the head delimits a female part in the form of a terminal conical socket (13), while the associated proximal end of the rod (9) consists of a complementary male conical part (18), designed to be introduced into the socket.

11. Prosthesis according to claim 10, characterized in that the fixing means comprise a screw (19) designed to be screwed into a corresponding tapped hole formed axially in the male conical part (18), means (16) being provided to allow the screw to be introduced into the said tapped hole.

12. Prosthesis according to claim 11, characterized in that the said means consist in a passage arranged in the head (12) axially with respect to the socket (13) so as to allow the screw (19) to be placed therein.

13. Prosthesis according to claim 9, characterized in that the said metaphysal part (33) delimits a conical male part (34) and the proximal end of the rod (39) consists of a female part (38) forming a conical housing (41) designed to accommodate the said male part.

14. Prosthesis according to claim 13, characterized in that the said means of fixing the male (34) and female (38) parts comprise a screw (37) designed to be introduced into an axial bore (35) of the conical male part (34) and to be screwed into a tapped hole (42) arranged in the proximal end (38) of the rod (39) axially beyond the conical housing (41).

15. Prosthesis according to claim 14, characterized in that an opening (36) is formed in the metaphysal part (33) axially with respect to the bore (35) of the male part (34) so as to allow the screw (37) to be introduced into this bore.

16. Prosthesis according to claim 9, characterized in that a circular groove (26) is arranged around the conical male part (27) together with a conical transverse bore (28) in the corresponding female part (29), this bore opening into the groove roughly tangentially, and in that a conical pin (31) for immobilizing the said female and male parts is provided for insertion into the bore.

17. Prosthesis according to claim 6, characterized in that the metaphysal part (11, 33) is of one piece with the hemispherical head (12), it being possible for the assembly to be secured with the rod (9, 39).

18. Prosthesis according to claim 2, characterized in that it consists of three parts which can be assembled by appropriate means, namely the hemispherical head (12), the metaphysal part and the humerus rod (9, 39).

19. Set of shoulder prostheses, according to claim 1, characterized in that it comprises one or more humerus rods and one or more humerus heads, the dimensions of the rods and/or the heads being different.

20. Try-in prosthesis able to be used for fitting prostheses according to claim 1, characterized in that it possesses a try-in rod (43) corresponding to the try-in rod of the actual prosthesis, and a head (44) corresponding to the head of the actual prosthesis, the said head carrying a shim (46) of a determined thickness able to come into contact with an end of the shaft of the humerus so as to determine the size of the actual prosthesis.

21. Try-in prosthesis according to claim 20, characterized in that the try-in head (44) is removable and can be fitted onto and removed from the try-in rod (43) and the prosthesis rods (9, 39).

22. Method for fitting a shoulder prosthesis (5, 32), comprising a humerus rod (9, 39) intended to be introduced into the medullary cavity of a fractured humerus (2′), and a roughly hemispherical head (12) which can be secured to the humerus rod and is designed to become lodged in a glene of the shoulder, it being possible for the said head to be fixed on the humerus rod in any radial plane with respect to the axis of the humerus rod (9, 39), comprising the steps of positioning the humerus rod at the appropriate height in the medullary cavity, sealing the said rod into the said cavity, fixing the shoulder muscles on the head, positioning the bony fragments on the head, mounting the head onto the sealed rod, adjusting the retroversion of the head in rotation with respect to the rod, then permanently fixing the head on the rod.

23. Method according to claim 20 in which the shoulder prosthesis is a prosthesis according to claim 1

24. Prosthesis according to claim 2, characterized in that it comprises fixing means being peripheral holes (20) in the head for fixing elements of the cuff.

25. Prosthesis according to claim 2, characterized in that the humerus head is secured to a metaphysal part (11, 13) which can be assembled with the humerus rod in any angular position.

26. Prosthesis according to claim 2, characterized in that the humerus head forms a separate part from a metaphysal part capable of being assembled with the humerus head in any angular position.

27. Prosthesis according to claim 2, characterized in that the means of fixing on the humerus head comprise an assembly of male and female elements (13, 18, 34, 38) allowing fixing, after the head has been positioned, in any relative angular position whatsoever.

28. Prosthesis according to claim 2, characterized in that the humerus head and the humerus rod are assembled by means of a male-female system, particularly of the Morse taper type.

29. Prosthesis according to claim 28, characterized in that a metaphysal part (11) secured to the head delimits a female part in the form of a terminal conical socket (13), while the associated proximal end of the rod (9) consists of a complementary male conical part (18), designed to be introduced into the socket.

30. Prosthesis according to claim 29, characterized in that the fixing means comprise a screw (19) designed to be screwed into a corresponding tapped hole formed axially in the male conical part (18), means (16) being provided to allow the screw to be introduced into the said tapped hole.

31. Prosthesis according to claim 30, characterized in that the said means consist in a passage arranged in the head (12) axially with respect to the socket (13) so as to allow the screw (19) to be placed therein.

32. Prosthesis according to claim 28, characterized in that the said metaphysal part (33) delimits a conical male part (34) and the proximal end of the rod (39) consists of a female part (38) forming a conical housing (41) designed to accommodate the said male part.

33. Prosthesis according to claim 32, characterized in that the said means of fixing the male (34) and female (38) parts comprise a screw (37) designed to be introduced into an axial bore (35) of the conical male part (34) and to be screwed into a tapped hole (42) arranged in the proximal end (38) of the rod (39) axially beyond the conical housing (41).

34. Prosthesis according to claim 33, characterized in that an opening (36) is formed in the metaphysal part (33) axially with respect to the bore (35) of the male part (34) so as to allow the screw (37) to be introduced into this bore.

35. Prosthesis according to claim 28, characterized in that a circular groove (26) is arranged around the conical male part (27) together with a conical transverse bore (28) in the corresponding female part (29), this bore opening into the groove roughly tangentially, and in that a conical pin (31) for immobilizing the said female and male parts is provided for insertion into the bore.
